(19) United States
(12) Patent Application Publication
(10) Pub. No.: US 2002/0160932 A1
(43) Pub. Date: Oct. 31, 2002

(54) Uncompetitive inhibitors of helicase-primase

(76) Inventors: Gerald Kleymann, Wuppertal (DE); Rudiger Fischer, Pulheim (DE); Martin Hendrix, Odenthal (DE); Guy Hewlett, Wuppertal (DE); Veniamin Pevzner, Berlin (DE); Ulrich Betz, Wuppertal (DE); Wolfgang Bender, Wuppertal (DE); Peter Eckenberg, Wuppertal (DE); Gabriele Handke, Wulfrath (DE); Udo Schneider, Leverkusen (DE); Olaf Weber, Woodbridge, CT (US); Kerstin Henninger, Wuppertal (DE); Axel Jensen, Velbert (DE); Jorg Keldenich, Wuppertal (DE); Judith Baumeister, Wuppertal (DE)

Correspondence Address: Jeffrey M. Greenman, Bayer Corporation, 400 Morgan Lane, West Haven, CT, 06516, US

(21) Appl. No.: 09/943,106

(22) Filed: Aug. 30, 2001

(30) Foreign Application Priority Data
Sep. 7, 2000 (DE) 10044353.2

(51) Int. Cl. A61K 31/00; A61K 48/00; A01N 61/00; A61K 31/70; A01N 43/04; A61K 39/245; A61K 39/255; A61K 39/265; A61K 39/27

(52) U.S. Cl. 514/1; 514/44; 424/229.1

(57) ABSTRACT

The present invention relates to inhibitors of the viral helicase-primase enzyme complex with anti-herpes activity, and to medicaments containing these compounds in suitable formulation. The invention further describes a method for identifying substances with antiviral activity in order to treat herpes infections in humans and animals.

The present invention relates to inhibitors of the viral helicase-primase enzyme complex with anti-herpes activity, and to medicaments containing these compounds in suitable formulation. The invention further describes a method for identifying substances with antiviral activity in order to treat herpes infections in humans and animals.

The action of the compound is essentially based on the uncompetitive inhibition of viral helicase-primase, the simultaneous binding of the substances to the helicase and the primase subunits of the enzyme complex, the inhibition of virus replication in vitro and in vivo and, last but not least, the very good pharmacokinetic and pharmacodynamic properties which lead to an effect not previously achieved in the animal model.

Descriptions are given hereinafter of the specific aspects such as the viral replication assay in cell culture, the binding of a molecule to two targets, the herpes viruses, and related research, the state of the art and the invention itself.

This application gives a description of a viral replication assay which permits the identification of effective and tolerated antiviral compounds. The strategy is based on simultaneous selection of molecules according to 2 essential criteria, specifically in the first place with reference to the antimicrobial effect (the molecule binds functionally to a relevant target (positive selection)), and in the second place the compounds must be tolerated, that is to say the molecules do not then bind or modulate any unwanted (host) targets (negative selection).

Numerous medications and therapies have been invented and, in the recent past, there has been increasing combination of these methods in order to increase the therapeutic efficacy, because monotherapies have in some cases not shown the desired profound benefits on administration to patients.

Unfortunately, on use of the combination therapy there has been found in some cases to be, besides the desired potentiation of the therapeutic effect, also an increase in side effects attributable to low tolerability. A conventional example of the combination therapy which may be mentioned here is the treatment of HIV-infected patients or the therapeutic regimen for AIDS patients.

In the area of antiinfective research, for example, an active substance which simultaneously inhibits two essential targets in the reproduction cycle of a pathogen may display an enhanced therapeutic effect. This enhancement of effect is based on cumulative inhibitory effects as have been shown in the past for the analogous case of combination therapy, which is frequently superior to the monotherapy (one-molecule chemotherapy) in which the active substance in the therapeutic composition usually modulates only one relevant binding cavity of a protein or a subunit of a protein complex. If the active substance binds in the region of the contact site of two targets, frequently stronger binding may be found, owing to avidity effects. The better binding properties and/or the cumulative inhibitory effects result in a superior therapeutic action, which ideally is displayed without side effects, that is to say is distinguished by being very well tolerated. This means the pleasing consequence for the patient of lower doses.

The Herpesviridae developed over millions of years of evolution and are very widespread in nature. Members of this family have been detected in humans, nonhumanoid primates and most other mammals and vertebrates (Virology, 1996, Fields et al. Lippincott-Raven Publishers, Philadelphia, Pa. 19106, USA). The herpesviruses are enveloped, double-stranded DNA viruses which infect permissive cells which carry on their cell surface besides negative charges from heparan sulphate and/or glycosaminoglycans in addition the so-called herpes viral entry mediator. A remarkable property of the viruses is their ability to develop a life-long latency in the infected host and to reactivate, and induce recurrent infections, more or less frequently from the pool of latently infected cells in the event of endogenous or external stimuli.

Eight human herpesviruses (HHV1 to HHV8) have been described to date. The viral genomes (˜125-230 kbp of information) have been sequenced and deposited in the usual databases, which are also accessible through the Internet (GenBank, EMBL database etc.). High sequence homologies have been identified on comparison of the genomes. This relates in particular to the genes encoding helicase and primase; this means that the replication machinery is highly conserved among herpes viruses but can clearly be distinguished from the host's DNA replication on comparison with eukaryotic genes. The genomes code for more than 50 reading frames which are essential for the replication cycle of the viruses in vitro or in vivo. Numerous publications discuss the relevance or the suitability of these potential targets for successful antiviral therapy. The function of the helicase-primase enzyme complex in the replication cycle and its suitability as target for an efficient antiviral (chemo)therapy of a herpes viral infection has also been published (Herpes Simplex Virus Replication, 1997, Annual review of Biochemistry, Boehmer PE & Lehmann IR, 66, 347-384; The Structure and function of the HSV DNA replication proteins: defining novel antiviral targets, 1993, Antiviral Research, Matthews J T, Terry B J, Field A K, 20, 89-114).

The herpesviruses have been divided on the basis of similar biological properties into 3 subfamilies, namely α (HHV1 to 3), β (HHV5 to 7), and γ (HHV4 and HHV8) herpesviruses. Trivial names have been derived on the basis of the clinical symptoms of the pathological state or simply for historical reasons as follows: herpes simplex virus 1 and 2 (HSV-1 and -2 cause herpes labialis and genitalis) also represent HHV1 and 2; varicella zoster virus (VZV causes chickenpox and shingles) is used synonymously with HHV3; and Epstein-Barr virus (EBV) and cytomegalovirus (HCMV) are used synonymously with the designation HHV4 and 5.

The proportion of those infected by one or more herpes viruses within a population is between fewer than 10 and more than 90%, depending on the age group, the sex, the social status, geographical aspects and, of course, on which herpesvirus is investigated. Precisely because some herpesviruses are ubiquitous pathogens which cause a number of diseases which may extend from mild symptoms which impair only normal daily activities, up to sight or life-threatening diseases—particular mention may be made here of keratitis in immunosuppressed patients, a generalized viraemia, or a retinitis in AIDS patients, miscarriage in pregnant women, and hepatitis, encephalitis or deafness in neonates—there is a strong medical demand for a safe and effective medication, and wide-ranging research programmes have been set up to find novel, more effective virus therapeutic agents.

Zovirax™ (acyclovir), a selective and specific inhibitor of herpes virus replication, was the first milestone, at the end of the 1970s, in the development of antiviral active substances. Novel derivatives of this very commercially successful nucleoside are penciclovir or the pharmacokinetically optimized prodrugs valacyclovir and famciclovir, which provide the patient with a more convenient dose or therapeutic regimen, were launched in the mid to late 1990s. Nucleosides are present, for lack of an alternative, in fact the therapeutics of choice for herpes infections. The nucleosides are, however, so-called prodrugs which must first be phosphorylated by viral thymidine kinase (TK) and then subsequently converted by cellular kinases so that the actual active substance, the nucleoside triphosphate, can inhibit the activity of the viral DNA polymerase. If the virus has no functionally active TK, as is the case, for example, with resistant HHV1 mutants, with TK-negative viruses such as HCMV or viruses which do not have an optimal primary structure in relation to viral DNA polymerase, the active substance is unable to display its full potential, the selectivity index is markedly reduced and higher doses must be administered, which may in turn lead to increased side effects. Since nucleosides and nucleotides are obligate or non-obligate terminators of DNA polymerization, they have a mutagenic potential, which has been comprehensively documented for the nucleoside Cymevene™ (ganciclovir). In summary, it can be stated that a possible broad-spectrum anti-herpes activity, efficacy of the medicaments especially when therapy is initiated late, drug safety and the occurrence of nucleoside-resistant viruses clearly outline the objectives aimed at for the next generation of therapeutics based on novel mechanisms of action.

A novel option, the ternary herpes helicase primase complex consisting of UL5, UL8 and UL52, is known (Ann. Rev. Biochem. 1990, 59, 289-329), as is its suitability as anti-herpes target (Antiviral Research 20, 1993, 89-114).

Inhibitors of herpes helicase-primase are likewise disclosed in J. Med. Chem. 1995, 38, 1811-1819.

The Patent Application WO 97/24343 describes a method for identifying inhibitors with anti-herpes properties, which is characterized in that compounds which, if they bind to the DNA-helicase-primase complex, stabilize the DNA-enzyme complex are selected. It is assumed that these inhibitors bind to allosteric binding sites on the UL5 or UL52 gene product of the HSV helicase-primase or homologous herpes viral enzyme complexes, and thus induce the stabilizing effect. This allosteric binding cavity is thought to be localized in the region of the A-B sequence on the UL52 subunit, and the inhibition is mediated by blocking a terminal zinc finger on a catalytic subunit of the herpes helicase-primase. A crucial disadvantage of this method is that it frequently identifies compounds which do not have the necessary selectivity index in vitro or the required tolerability in vivo, consequently usually cannot be employed for the treatment of herpes infections because drug safety is not ensured.

The Application WO 99/42455 and the publication by Spector, F. C., Journal of Virology 1998, Vol 72, No 9, pages 6979-6987 describe compounds which bind exclusively to the UL5 subunit of the helicase-primase enzyme complex, which is demonstrated by wide-ranging mutation analysis of at least 25 different UL5 mutants. It is additionally found that, for example, T157602 as representative of this class of compound does not displace either ATP or DNA competitively from the enzyme complex. Substituted thiazolylphenyl derivatives are, remarkably, also described as essential and central structural motif for the compounds in the Application WO 97/24343 cited above.

Although the helicase-primase complex has been known for some time and there is a pressing need for novel antiviral targets (Antiviral Research, 20, 1993, 89-114), it has not to date been possible, despite various patent applications in this area, to identify any class of suitable compounds which could have reached the marketing stage. Besides lack of activity, this is in large part attributable to the occurrence of severe side effects.

The aim of this invention is therefore to provide a method with which not only is it possible to identify alternative or more effective compounds, but it is possible in particular to find active substances with an improved selectivity index in vitro and a superior tolerability in relation to the state of the art. A further aspect of this invention is the identification and provision of active substances which can be employed for the therapy of infections caused by nucleoside/nucleotide-resistant herpes viruses.

It has now been found, surprisingly, that medicaments which have the features and properties listed hereinafter and inhibit the herpes helicase-primase complex in a newly found way which is described herein are suitable for the treatment of herpes infections.

The present invention solves the problem described above by providing medicaments which contain uncompetitive inhibitors of the herpes helicase-primase.

In another preferred embodiment, the medicaments are also characterized in that the helicase-primase is at least 50% homologous with the helicase-primase of herpes simplex virus 1, based on the nucleotide sequence or the amino acid sequence, whichever is higher.

In another preferred embodiment, the medicaments are characterized in that the helicase-primase-harbouring virus is herpes simplex virus 1.

In another preferred embodiment, the medicaments are characterized in that the compounds selected to be present in the medicament have the ability to bind simultaneously to the helicase and the primase subunit of the helicase-primase complex.

In another preferred embodiment, the medicaments are characterized in that the compounds selected to be present in the medicament have the ability to inhibit the DNA-dependent NTPase activity of the helicase-primase.

In another preferred embodiment, the medicaments are characterized in that the compounds selected to be present in the medicament have the ability to inhibit the replication of a herpes virus.

In another preferred embodiment, the medicaments are characterized in that the compounds selected to be present in the medicament have the ability to inhibit the replication of a herpes virus in cell culture by at least 50% at a concentration of 10 μM.

In another preferred embodiment, the medicaments are characterized in that the compounds selected to be present in the medicament have the ability to inhibit the replication of a herpes virus in cell culture by at least 50% at a concentration of ≦500 nM.

In another preferred embodiment, the medicaments are characterized in that the helicase-primase-harbouring virus is PRV or BHV.

In a preferred embodiment there is description of a method for identifying compounds with anti-herpes activity, which is characterized in that the compounds are tested for their ability to inhibit helicase-primase, and those compounds which inhibit the helicase-primase enzyme complex uncompetitively are selected.

In another embodiment, the invention relates to compounds identified by any method claimed herein.

In another preferred embodiment, the invention is characterized in that the compound is not a nucleoside or nucleotide.

In another preferred embodiment, the invention relates to compounds used for producing medicaments for the treatment and prophylaxis of herpes infections.

In another preferred embodiment, the invention relates to medicaments or pharmaceutical formulations containing compounds in accordance with the methods detailed above.

In another preferred embodiment, the invention describes a method for the treatment and prophylaxis of herpes infections in mammals, which is characterized in that a therapeutically effective amount of the medicament claimed herein or of the pharmaceutical formulation is administered to the mammal requiring such a therapy.

In another preferred embodiment, the invention relates to medicaments which are characterized in that they contain compounds having the following general structural formula (I)

Z—Y—A—X—SO₂NR¹R² (I)

in which

Z represents (C₆-C₁₀)-aryl, which optionally can be substituted by 1-3 substituents, selected from (C₁-C₆)-alkanoyl, (C₁-C₆)-alkoxy, (C₁-C₆)-alkyl, halogen, (C₁-C₆)-alkoxycarbonyl, nitrohalogen (C₁-C₆)-alkyl, halogen(C₁-C₆)-alkoxy), amino(C₁-C₆)-alkylthio, hydroxy, carboxyl, carbamoyl, mono- or di-(C₁-C₆)-alkylaminocarbonyl, mono- or di-(C₁-C₆)alkanoylamino, (C₁-C₆)-alkoxycarbonyoamino, (C₁-C₆)-alkylsulfoxy, C₁-C₆-alkylsulfonyl, tri-(C₁-C₆)-alkylsilyloxy, tri- (C₁-C₆)-alkylsilyloxy, a 3- to 8-membered saturated or unsaturated, non-aromatic mono- or bicyclic heterocycle with up to 3 heteroatoms selected from the group S, N and/or O, which is optionally bonded via a nitrogen atom, and/or cyano,

or

Z represents a 5- to 6-membered aromatic heterocycle with up to 3 heteroatoms selected from the group S, N and/or 0, which is optionally bonded via nitrogen atom, which is optionally substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halogen-(C₁-C₆)-alkyl, halogen-(C₁-C₆)-alkoxy, amino, (C₁-C₆)-alkylthio, hydroxy, carboxyl, carbamoyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulfoxy, (C₁-C₆)-alkylsulfonyl, a 3- to 8-membered saturated or unsaturated, non-aromatic mono- or bicyclic heterocycle with up to 3 heteroatoms selected from the group S, N and/or O, which is optionally bonded via a nitrogen atom, and/or cyano,

or

Z represents a 3- to 8-membered saturated or unsaturated, non-aromatic, mono- or bicyclic heterocycle, up to 3 heteroatoms selected from the group S, N and/or O, which is optionally bonded via a nitrogen atom, which can optionally be substituted by 1 to 3 substituents selected from oxo, halogen, hydroxy, (C₁-C₆)-alkoxycarbonyl, (C₁-C₆)-alkoxycarbonylamino, (C₁-C₆)-alkyl, halogen-(C₁-C₆)-alkyl and hydroxy-(C₁-C₆)-alkyl,

or

Z represents —OR¹⁹, —NR²⁰R²¹ or —CO—NR²²R²³,

wherein

R¹⁹ represents phenyl, which optionally can be substituted by a group of the formula —NR²⁴R²⁵, wherein

R²⁴ and R²⁵ are identical or different and represent hydrogen, (C₁-C₆)-alkyl or (C₁-C₆)-acyl,

or

R¹⁹ represents (C₁-C₆)-alkyl or (C₃-C₈)-cycloalkyl, which optionally can be substituted 1- to 3-fold by hydroxy and/or halogen,

R²⁰ and R²¹ are identical or different and represent hydrogen, carbamoyl, mono- or di-(C₁-C₆)-alkylaminocarbonyl, phenyl, (C₁-C₆)-acyl or (C₁-C₆)-alkyl,

wherein (C₁-C₆)-alkyl optionally can be substituted by (C₁-C₆)-alkoxy, (C₁-C₆)-acyl, phenyl or a 5 to 6-membered aromatic heterocycle with up to 3 heteroatoms selected from the group S, N and/or O,

wherein phenyl and aromatic heterocycle optionally can be substituted 1- to 3-fold identically or different by halogen and/or hydroxy,

R²² and R²³ are identical or different and represent hydrogen or (C₁-C₆)-alkyl,

Y represents (C₆-C₁₀)-aryl, a 5- to 6-membered aromatic heterocycle, a 5- to 6-membered aromatic benzo-fused heterocycle, a 3- to 8-membered saturated or unsaturated, non-aromatic heterocycle which is optionally bonded via a nitrogen atom,

and which optionally can be substituted with 1 to 3 substituents selected from the group consisting of

halogen, (C₁-C₆)-alkoxy, (C₁-C₆)-alkoxycarbonyl, (C₁-C₆)-alkylthio, hydroxy, carboxyl, partially fluorinated (C₁-C₆)-alkoxy with up to 6 fluorineatoms, (C₁-C₆)-alkyl,

A represents a group of the formula —CR^{4′}R^{5′}—C(O)—CR⁴R⁵—, —CR^{4′}R^{5′}—C(O)—NR⁹—, —NR^{9′}—C(O)—CR⁴R⁵— or —NR^{9′}—C(O)—NR⁹—,

in which

R⁴, R^{4′}, R⁵, R^{5′}, R⁹ and R^{9′} are identical or different and represent hydrogen, (C₁-C₆)-alkyl, (C₂-C₆)-alkenyl or (C₃-C₈)-cycloalkyl,

X represents (C₆-C₁₀)-aryl, a 5- to 6-membered aromatic heterocycle, a 5- to 6-membered aromatic benzo-fused heterocycle, a 3- to 8-membered saturated or unsaturated, nonaromatic heterocycle which is optionally bonded via a nitrogen atom,

R¹ and R² are identical or different and represent hydrogen, (C₁-C₆)-alkyl or (C₃-C₈)-cycloalkyl,

where the A—X—SO₂ angle is 153°±15%, and the compound has a molecular mass of less than 500 g/mol,

and the salts thereof.

The invention set forth herein solves the problems detailed above and meets the set aims for novel therapeutic agents by describing methods for identifying novel (non-nucleosidic) active substances which directly inhibit the enzymatic activity of the herpes helicase-primase complex in a novel way which is described here for the first time. The inhibition of this essential enzyme complex when the compound has a suitable structure, which is disclosed herein by a generic pharmacophore model in addition to active substances specified by way of example, leads not only to inhibition of DNA replication but also and in particular to inhibition of herpesvirus replication in vitro and in vivo. In addition, precisely because the helicase-primase enzyme complex is highly conserved in the Herpesviridae family, it has been possible for the present invention to show a broad-spectrum anti-herpesvirus activity of the compounds (Table 1). The selective effect of the inhibitors on members of the herpesvirus family and, in particular, on acyclovir-resistant herpesviruses, in combination with a suitable safety profile make these active substances an agent which has been desired for some years for the treatment of herpes infections.

The term “herpes” used in the context of this invention includes all viruses of the family of herpes viruses and encompasses in particular the herpesviruses which code for helicase-primase proteins. This applies in particular to proteins which are homologous in relation to the herpes helicase-primase of HSV. This applies in particular to those where the helicase or primase is at least 50% homologous with the helicase or primase of herpes simplex virus 1, based on the nucleotide or amino acid sequence, whichever is the higher. The herpesvirus family includes, for example, HHV-1 to HHV-8, EHV, BHV, PRV etc.

The herpes simplex viruses 1 and 2 are designated according to their serotype which are characterized on the basis of specific monoclonal antibody reactions.

The term helicase-primase refers to the helicase-primase enzyme complex which is necessary for replication of viral DNA. In the case of herpes simplex viruses, this enzyme complex consists of the proteins of the UL5, UL8 and UL52 genes or at least the (necessary or essential) UL5 and UL52 gene products; in the case of other members of the herpes family, reference will be made to the corresponding homologues.

The term helicase designates the subunit of the helicase-primase complex which is involved in viral DNA replication. In the case of the herpes simplex viruses, this is the UL5 gene product; in the case of other members of the herpes family, reference will be made to the corresponding homologues.

The term primase designates the subunit of the helicase-primase complex which is involved in viral DNA replication. In the case of herpes simplex viruses this is the UL52 gene product; in the case of other members of the herpes family, reference will be made to the corresponding homologues.

The abbreviation moi stands for “multiplicity of infection” and is the quotient of the number of infectious particles or cells of the pathogen divided by the number of cells to be infected.

(MOI=number of cells or number of infectious particles of the pathogen/number of cells which are infected).

The term IC₅₀ or EC₅₀ describes the concentration or the amount of active substance required to achieve 50% inhibition in a given test system or the effective concentration required to achieve a half-maximum signal in an assay.

The term selectivity index (SI) refers to the quotient of the concentrations at which the cell vitality is reduced to 50% compared with the cell control, divided by the concentration at which the antimicrobial effect reaches at least 50% of a maximally possible inhibition of pathogen replication, that is to say SI=(CC₅₀/IC₅₀). The larger this number is, the greater is the tolerability of the compound. Compounds with SI values greater than 10 are preferred.

The term therapeutic index (TI; tolerability) refers to a quotient of the lethal dose (LD) at which 50% of the experimental animals die owing to effects of the substance, and the effective dose (ED) at which 50% of the experimental animals survive the infection, TI=(LD₅₀/ED₅₀).

The term formulation in relation to active substances describes a pharmaceutical composition consisting of non-toxic, generally inert excipients and carrier materials which cause no adverse effect on the action of the active ingredient but, on the contrary, permit optimal administration of the active substance in relation to pharmacokinetics and pharmacodynamics.

The term to inhibit describes, when used in connection with the enzymatic activity, the inhibition of the maximally possible enzymic activity by at least 50% at a concentration of about IC₅₀=100 μM or less and preferably at a concentration which is as low as possible in an appropriate in vitro assay employed for determining the enzymatic activity.

The term uncompetitive inhibition describes a rare type of inhibition in which the inhibitor binds to the enzyme-substrate complex. With this type of inhibition there is a change in the maximum rate but Km remains unchanged within the uncertainty of the experiment. The term uncompetitive expressly includes partially uncompetitive types of inhibition.

The term to inhibit describes, when used in connection with the in vitro viral replication assay, generally the inhibition of microbial growth by about 50% at a concentration of IC₅₀=100 μM or less and preferably a concentration which is as low as possible in order to reach the value.

The term target usually refers herein to the target molecule whose activity is modulated where appropriate by active substances. Generally speaking, the target is frequently a gene product which is essentially involved in the development of a pathological state.

The term mutation in a target describes a mutation in a gene which leads to an alteration in the amino acid sequence of the corresponding protein.

The term reduction in relation to the loss of increase or reduction in the activity is used when the change in activity is significant, more favourably reaches a factor of 2 and preferably changes by an order of magnitude.

(C₁-C₆)-Alkyl expediently represents a straight-chain or branched alkyl radical having 1 to 6 carbon atoms. A straight-chain or branched alkyl radical having 1 to 4 carbon atoms (C₁-C₄) is preferred. Examples which may be mentioned are: methyl, ethyl, n-propyl, isopropyl, n-butyl, sec-butyl, tert-butyl, n-pentyl and n-hexyl. A straight-chain or branched alkyl radical having 1 to 3 carbon atoms ((C₁-C₃)alkyl) is particularly preferred.

(C₆-C₁₀)-Aryl generally represents an aromatic radical having 6 to 10 carbon atoms. Preferred aryl radicals are phenyl and naphthyl.

(C₃-C₈)-Cycloalkyl represents for the purposes of the invention cyclopropyl, cyclopentyl, cyclobutyl, cyclohexyl, cycloheptyl or cyclooctyl. Those which may be mentioned as preferred are: cyclopropyl, cyclopentyl and cyclohexyl. The meaning of (C₃-C₆)-cycloalkyl accordingly expediently represents cyclopropyl, cyclopentyl, cyclobutyl, cyclohexyl.

A 5- to 6-membered aromatic heterocycle with up to 3 heteroatoms from the series S, O and/or N represents, for example, pyridyl, pyrimidyl, thienyl, furyl, pyrrolyl, thiazolyl, N-triazolyl, oxazolyl or imidazolyl. Pyridyl, furyl, thiazolyl and N-triazolyl are preferred.

A 5- to 6-membered aromatic benzo-fused heterocycle having up to 3 heteroatoms from the series S, O and/or N represents, for example, benzimidazolyl.

A 3- to 8-membered saturated or unsaturated, nonaromatic heterocycle which is optionally bonded via a nitrogen atom and has up to 3 heteroatoms from the series S, N and/or O includes, for example, morpholinyl, piperidinyl, piperazinyl, methylpiperazinyl, thiomorpholinyl or pyrrolidinyl, as well as 3-, 7- and 8-membered heterocycles such as, for example, aziridines (for example 1-azacyclopropan-1-yl), azetidines (for example 1-azacyclobutan-1-yl) and azepines (for example 1-azepan-1-yl). The unsaturated representatives may contain 1 to 2 double bonds in the ring.

The compounds described in this patent application, and the pharmacophore formula and the mechanism of action of the substances are novel and can be clearly distinguished in particular from the Applications WO 97/24343, WO 99/42455 and all publications published up to the priority. Whereas the substances disclosed in the Application WO 97/24343 bind to an allosteric binding site on the UL5 or UL52 gene product of the HSV helicase primase, and thus enhance or stabilize the binding of enzyme complex to the DNA substrate (according to the authors, this allosteric binding site is located within the so-called A-B sequence on the UL52 subunit and the inhibitory effect is caused by modulation of a terminal zinc finger motif on a catalytic subunit of the herpes helicase-primase complex), the substances published in the Application WO 99/42455 bind exclusively to the UL5 subunit of the complex, which is explicitly stated in the publication by Spector, F. C., Journal of Virology 1998, Vol 72, No 9, pages 6979-6987.

Suitable compounds which inhibit the helicase-primase complex in accordance with the abovementioned mechanism can be identified by testing the ability of the substances to bind to the helicase (subunit) or the primase (subunit) of the helicase-primase complex. It is preferred to test the ability of the active substances to inhibit the enzyme-associated DNA-dependent NTPase activity of the herpes helicase-primase (such as, for example, helicase-primase of the herpes simplex virus), which is advantageously carried out technically by means of high-throughput screening, HTS). An alternative possibility is also to test the ability of the substances to inhibit viral replication in an in vitro culture system, and subsequently to select the substances which uncompetitively inhibit the DNA-dependent hydrolysis of relevant nucleotides.

Numerous methods have been described for direct measurement of the binding of molecules to proteins, but many of these test systems require, in order to obtain quantitative results, purified protein or at least an enriched fraction. Mention should be made at this point of the fact that not all molecules binding to the protein or enzyme modulate the function of the target in the desired manner.

The enzymatic assay is simple to carry out, but the enzyme must be purified functionally from a herpes-infected cell culture. An alternative possibility is for the genes of the enzyme complex to be cloned by methods of genetic manipulation into an appropriate expression system and then expressed. If there is functional production of the enzyme, it must be enriched or, preferably, prepared in pure form by conventional purification techniques.

A viral replication assay can be carried out very simply as described in this application in a cell culture system. This assay is possibly more sensitive than the enzymatic assay systems mentioned because virus replication in cell culture is the best simulator of the actual infectious event and, if appropriate, the number of enzyme molecules is lower in relation to the inhibitor than in the enzyme assay. If inhibitors of virus replication are identified, it is, however, necessary to use other methods such as, for example, the preparation of mutants with subsequent genome sequencing in combination with a complementation analysis in order to elucidate the mechanism of action or to identify the underlying target (in the present example the viral helicase-primase).

Active substances which show an effect in the enzyme assay or which inhibit virus replication in the cell culture system can be characterized in more detail in a test which permits conclusions to be drawn about the binding properties of the molecule to the helicase-primase complex. On the other hand, compounds which elicit an effect in the binding assay can be functionally investigated in more detail subsequently by means of an enzyme assay or be tested for inhibition of virus replication in cell culture. Even if the test systems described hereinafter are listed in a possible sequence, this is not intended to mean that all the tests for successful identification of herpes helicase-primase inhibitors must be carried out in exactly this sequence but, on the contrary, is intended to mean that the number of different tests as well as their sequence lies within the description of the skilled person in order to reach the target efficiently.

Other test systems in turn allow it to be established whether a given active substance inhibits enzyme-mediated RNA primer biosynthesis or whether the substance modulates the helicase activity of the complex.

DNA, and preferably single-stranded DNA with an appropriate primase concensus sequence in the case of the primase assay or a DNA substrate which is able to form a replication fork of similar structure, can be employed in the helicase assay. Numerous options have been described and reference may be made at this point to the review literature (Boehmer P E & Lehmann I R Awn Rev Biochem 1997) (66) 347-84 or the specific publications (High-Throughput Screening Assay for Helicase Enzymes, 1998, Analytical Biochemistry, M Sivaraja, H Giordano, M G Peterson, 265, 22-27).

Preferred or suitable compounds described in this application do not intercalate into nor bind in another way directly to double-stranded DNA.

It is possible in all the methods described above for the (non-nucleosidic) compound also to be tested for the ability to inhibit the herpes helicase-primase-mediated RNA primer biosynthesis or the helicase activity. It is, however, crucial that the (non-nucleosidic) active substances described in this invention are tested for their ability to inhibit virus replication in cell culture by at least 50% at a concentration of less than 10 μM. The compounds preferably selected are those which inhibit virus replication at concentrations which are as low as possible. However, it must be taken into account in this connection that these compounds also have the desired pharmacokinetic and pharmacodynamic properties.

It should also be mentioned that the compounds, the formulated active substances and the disclosed methods are suitable for successful treatment of herpes infections which do not respond to a nucleoside therapy and are usually caused by nucleoside-resistant herpesviruses.

This aspect of the invention of course includes the method of treating acyclovir-resistant herpes infections by administration to the infected mammal of effective doses of one of the disclosed compounds, which may be formulated appropriately if necessary.

It has been possible by use of the methods described above to identify inhibitors of herpes helicase-primase successfully. The specific and selective effect of the compounds on herpesviruses in combination with an appropriate safety profile make the active substances the desired agent for future treatment of herpes infections.

Anti-herpes Activity and Test Systems

The antiviral activity of the compounds can be shown by biochemical, microbiological and biological methods.

This invention discloses the inhibition of viral replication of herpes viruses in cell culture, which is shown by way of example for members of the herpes family such as HSV, BHV and PRV (see Table 1).

The HTS capable viral replication assay to be carried out in cell culture provides in a more efficient manner data analogous to the so-called plaque reduction assay and is described hereinafter.

A biochemical method for demonstrating the activity of the compound on the viral helicase-primase target is described in the biochemical assay section. This ATPase assay is only one of the numerous options which are available to the skilled person and are detailed, for example, in “High-Throughput Screening Assay for Helicase Enzymes, 1998, M. Sivaraja, H. Giordano and M. G. Peterson, Analytical Biochemistry, 265, 22-27”. Further assay systems which are detailed in diverse configurations therein are suitable for establishing whether, where appropriate, compounds inhibit directly the helicase or the primase activity of the helicase-primase complex.

Detection of a therapeutic effect of the compounds is carried out using an in vivo model. The so-called lethal challenge animal model, and the doses for administration and therapeutic regimens, are summarized in detail below.

Description of the Tables

Table 1 summarizes the data on inhibition of the helicase-primase enzyme complex (Kᵢ values), results on the mechanism of action, the IC₅₀ and EC₅₀ data on inhibition of virus replication of the wild-type and therapy-resistant HSV strains, the ED₅₀ values for wild-type virus in the animal model and the relevant mutation and cross-resistance patterns.

Direct comparison of the IC₅₀ values obtained in cell culture shows that some selected compounds are more potent by at least one order of magnitude than the market standard acyclovir (Zovirax™). The data obtained in vivo clearly show that this action of the compounds found in vitro is reflected by a superior potency and efficacy in the animal model. The exemplary compounds inhibit acyclovir-resistant HSV-1 (F) mutants and wild-type HSV-1 (F) with virtually identical IC₅₀ values. Broad-spectrum anti-herpes activity is shown by the selected compounds of Examples 1, 4, 5, 6 and 7, which thus inhibit various herpes viruses such as, for example, the human herpes simplex virus as well as the porcine (PRV) and bovine (BHV) animal herpesvirus. These compounds are also active on all the clinical isolates tested to date (39 HSV-1 and 19 HSV-2 isolates have been tested). It is disclosed for the first time that compounds which meet the criteria of the features detailed above or, in other words, are covered by the claims detailed hereinafter are novel in relation to the state of the art and exceed the therapeutic standard in the animal model by at least one order of magnitude.

Assays and Test Systems

HSV-1 DNA-dependent ATPase assay (this in vitro assay permits testing for inhibition of the HSV-1 helicase-primase).

a) Preparation and purification of the enzyme

The HSV-1 helicase-primase heterodimer enzyme complex was [lacuna] with the aid of the recombinant baculovirus expression system in doubly infected Sf9 (Spodoptera frugiperda) cells. One recombinant baculovirus codes for the UL5 helicase gene and the second virus codes for the manipulated UL52-6×His primase gene.

The genes were amplified from the HSV-1 (F) genome by PCR (American Tissue Culture Collection ATCC VR-733) and cloned into the baculovirus (UL5→pFASTBAC1; UL52→pFASTHTb) as described in detail in the instruction manual for BAC-TO-BAC Baculovirus Expression Systems, Life-Technologies. The heterodimeric enzyme complex was purified using so-called IMAC chromatography as published in “Inhibition of Herpes Simplex Virus Replication by a 2-Amino Thiazole via Interactions with the Helicase Component of the UL-5-UL8-UL52 Complex”, 1998, Journal of Virology, F. C. Spector, L. Liang, H. Giordano, M. Sivaraja and M. G. Peterson, 74:6979-6987.

b) The ATPase assay

The purified heterodimeric helicase-primase complex (200 ng) was incubated with 1 μg of DNA (Sigma D3287 or D8681) in 20 mM HEPES (pH 7.6; 4-(2-hydroxyethyl)-1-piperazineethanesulphonic acid), 5 mM MgCl₂, 0.2-5 mM ATP, 100 μg of BSA (bovine serum albumin) per ml, 10% glycerol, 1 mM DTT (DL-dithiothreitol) and, where appropriate, inhibitor in the appropriate concentration at 37° C. for 60 min. The liberated inorganic phosphate was determined by colorimetry as published in “An improved assay for nanomole amounts of inorganic phosphate”, 1979, P. A. Lanzetta, I. J. Alvarez, P. S. Reinach and O. A. Candia, Analytical Biochemistry, 100:95-97.

The inhibition of the DNA-dependent ATPase activity was calculated from the change in the absorption in the presence and absence of inhibitor.

The enzyme kinetic analyses were carried out as described in the standard works of the literature such as, for example, Enzymkinetik, Theorie und Methoden [Enzyme Kinetics, Theory and Methods], by Hans Bisswanger, V C H Verlagsgesellschaft, 1994. The Kᵢ values were calculated with the assistance of the Sigma Plot software 4.0 and the method of nonlinear curve regression.

Table 2 shows the dose-dependent inhibition/titration of the ATPase activity of the HSV-1 helicase-primase enzyme complex with the representative Example 6. For this purpose, the ATPase activity of the helicase-primase heterodimer (detection through formation of inorganic phosphate (Pi)) was measured in the presence of saturated DNA concentrations and varying ATP and inhibitor concentrations, as explained in the ATPase assay section. The inhibition of the ATPase activity by the exemplary compound 6 is dose-dependent and is moreover influenced by the ATP concentration.

(The ATP control is carried out without enzyme and without DNA).

This can, of course, be proved better by comparison of the mathematical models for various mechanisms of inhibition. The experimental inhibition data in relation to the type of inhibition are best depicted by nonlinear regression based on the formulae for uncompetitive inhibitory behaviour.

Viral Replication Assay in Cell Culture

a) Viral replication and determination of the virus titre

HSV (HSV-1 Walki, HSV-1F or HSV-2G) was replicated on Vero cells (ATCC CCL-81) under the following conditions: the cells were grown in M199 medium (5% fetal calf serum, 2 mM glutarnine, 100 IU/ml penicillin, 100 μg/ml streptomycin) in cell culture bottles at 37° C. and 5% CO₂. The cells were split 1:4 twice a week. For the infection, the medium was removed, and the cells were washed with Hank's solution, detached with 0.05% trypsin, 0.02% EDTA (Seromed L2143) and incubated at a density of 4×10⁵ cells per ml under the abovementioned conditions for 24 hours. The medium was then removed, and the virus solution was added at an m.o.i of <0.05 in a volume of 2 ml per 175 cm² of surface area. After incubation under the conditions mentioned for one our, the medium was made up to a volume of 50 ml per 175 cm² bottle. Three days after the infection, the cultures showed distinct signs of a cytopathic effect. The virus was released by freezing (−80° C.) and thawing (37° C.) twice. The cell debris was removed by centrifugation (300 g, 10 min, 4° C.) and the supernatant was frozen in aliquots at −80° C.

The virus titre was determined by a plaque assay. For this purpose, Vero cells were seeded in a density of 4×10⁵ cells per well in 24-well plates and, after incubation (37° C., 5% CO₂) for 24 hours, infected with dilutions of the virus stock of from 10⁻² to 10⁻¹² (inoculum 100 μl). One hour after the infection, the medium was removed and the cells were covered with 1 ml of overlay medium (0.5% methylcellulose, 0.225 M sodium bicarbonate, 2 mM glutamine, 100 IU/ml penicillin, 100 μg/ml streptomycin, 5% fetal calf serum in MEM Eagle medium with Earle's salts) and incubated for 3 days. The cells were then fixed with 4% formalin for 1 hour, washed with water, stained with Giemsa (Merck) for 30 min and subsequently washed and dried. The virus titre was determined using a plaque viewer. The virus stocks used for the experiment had a titre of 1×10⁶/ml−1×10⁸/ml.

b) Measurement of the antiviral activity of test substances in the in vitro cell culture system

The anti-HSV effect was determined in a screening test system in 96-well microtitre plates with the assistance of various cell lines of neuronal, lymphoid and epithelial origin such as, for example, Vero (kidney cell line from the African green monkey), MEF (murine embryonic fibroblasts), HELF (human embryonic fibroblasts), NT2 (human neuronal cell line) or Jurkat (human lymphoid T-cell line). The effect of the substances on the spread of the cytopathogenic effect was determined by comparison of the reference substance acyclovir sodium (Zovirax®), a clinically approved anti-herpes chemotherapeutic agent.

The substances dissolved (50 mM) in DMSO (dimethyl sulphoxide) are investigated on microtitre plates (for example 96-well MTP) in final concentrations of 250-0.5 μM (micromolar) in duplicate determinations (4 substances/plate). For potent substances, the dilutions are continued over several plates down to 0.5 pM (picomolar). Toxic and cytostatic effects of the substances are also detected in these cases. After the appropriate dilutions (1:2) of the substances in medium on the microtitre plate, a suspension of cells (1×10⁴ cells per well), such as, for example, Vero cells in M199 (199 medium) with 5% fetal calf serum, 2 mM glutamine and optionally 100 IU/ml penicillin and 100 μg/ml streptomycin or MEF cells in EMEM (Eagle's minimum essential medium) with 10% fetal calf serum, 2 mM glutamine and optionally 100 IU/ml penicillin and 100 μg/ml streptomycin, or HELF cells in EMEM with 10% fetal calf serum, 2 mM glutamine and optionally 100 IU/ml penicillin and 100 μg/ml streptomycin, or NT2 and Jurkat cells in DMEM (4.5 mg/l glucose+pyridoxine) with 10% fetal calf serum, 2 mM glutamine, 1 mM sodium pyruvate, nonessential amino acids and optionally 100 IU/ml penicillin and 100 μg/ml streptomycin, is put in each well, and the cells in the relevant wells are infected with an appropriate amount of virus (HSV-1 F or HSV-2 G with an m.o.i (multiplicity of infection) of 0.0025 for HELF, Vero and MEF cells and with an m.o.i of 0.1 for NT2 and Jurkat cells). The plates are then incubated at 37° C. in a CO₂ incubator (5% CO₂) for several days. After this time, the cell lawn of, for example, Vero cells in the substance-free virus controls, starting from 25 infectious centres, completely lysed or destroyed (100% CPE) by the cytopathogenic effect of the HS viruses. The plates are initially evaluated visually with the aid of a microscope and then analysed with a fluorescent dye. For this purpose, the cell culture supernatant in all the wells of MTP is aspirated off and charged with 200 μl of PBS washing solution. The PBS is aspirated off once again and all the wells are charged with 200 μl of fluorescent dye solution (fluorescein diacetate, 10 μg/ml in PBS). After an incubation time of 30-90 min, the assay plates are measured in a fluorimeter with an excitation wavelength of 485 nm and an emission wavelength of 538 nm.

The IC₅₀ is the concentration of the inhibitor at which viral replication in cell culture is inhibited by 50% compared with the uninfected cell control or, alternatively, with an acyclovir control treated with a concentration which is 20 times the IC₅₀. The reduction in this case is determined through the fluorescence signal of the infected well of an MTP reaching 50% of the signal of the control detailed above.

The results obtained are summarized in Table 1.

c) Generation and sequencing of the resistant viral mutants

Naturally occurring, resistant viral mutants were selected in cell culture with the aid of the viral replication assay in the presence of 1 μm of exemplary substance 7 or a concentration which is at least 100 times the IC₅₀ for the other examples as listed in Table 1. 10 000 Vero cells are seeded in microtitre plates with 96 wells as described above and incubated overnight. The substance is added in the appropriate concentration, and the cells are then infected with 1 000 PFU (plaque forming units; m.o.i 0.1). Mutants occurred with a frequency of 1×10⁻⁶ to 5×10⁻⁶ in the case of HSV- 1 (F). The mutants were found by microscopic inspection or simply by freezing (−80° C.) 10 μl aliquots and subsequently analysing the 20-40 MTP with the fluorescent dye fluorescein diacetate as described above. If a resistant virus is present in a well, the corresponding fluorescence signal is reduced by a factor of at least 3 compared with a mutant-free well. The supernatants which tested positive or the frozen aliquots were used to grow larger quantities of the resistant virus and then determine the titre. The viral DNA was prepared in accordance with the protocols which are to be found in the handbook of methods “Herpes Simplex Virus Protocols, 1998, Ed. S. M. Brown & A. R. MacLean, Humana Press, Totowa, N.J.” and sequenced in accordance with the protocol “DNA sequencing with chain-terminating inhibitors, 1977, Sanger F, Nicklen S, Coulson A R, PNAS, 74, 5463-5467” using the ABI PRISM Big Dye Terminator kit from Applied Biosystems or using the DYEnamic ET terminator cycle sequencing kit from Amersham (apbiotech). Analysis of the samples was carried out in one of the conventional automatic sequencers.

The mutations of the resistant viruses found are compiled by way of example in Table 1.

TABLE 1
Pharmacological profile of the hlicase-primase inhibitors

| | | | | | | | | | | | | | in vivo | |
|---|---|---|---|---|---|---|---|---|---|---|---|---|---|---|
| | | | | | | | | | | | | | lethal challenge | |
| | in vitro viral replication assay | | | | | | | | | | | in vitro | model | |
| | | | Active substance- | | Acyclovir- | | | | | | | ATPase | HSV-1 | |
| | Wild-type | | resistant | | resistant | human | | porcine | | bovine | | inhibition | Walkie | |
| | HSV-1 (F) | | HSV-1 (F) mutants | | HSV-1 (F) | HSV-2 G | | PRV | | BHV | | wild-type | ED₅₀ | |
| | IC₅₀ | | IC₅₀ | | mutants | IC₅₀ | | IC₅₀ | | IC₅₀ | | HSV-1 (F) | [mg/kg] | HSV-2 |
| | [μm] | SI | [μm] | Mutation(s) | IC_{50 [μm]} | [μm] | SI | [μm] | SI | [μm] | SI | Ki [nM] | p.o. | MS |
| Example 1 | 0.75 | 350 | | | 0.75 | 1.5 | 167 | 4 | >63 | 1.5 | >167 | | 60 | weak |
| Mutant 1 | | | >250 | UL5 M355V | | | | | | | | | | |
| | | | | UL5 V6621 | | | | | | | | | | |
| | | | | UL52 A897T | | | | | | | | | | |
| Example 2 | 0.5 | 400 | | | 0.5 | 1.5 | 200 | | | | | | 26 | weak |
| Example 3 | 0.001 | 3E+05 | >250 | | 0.001 | 0.005 | 25000 | | | | | | 8 | 11 |
| Example 4 | 0.0005 | 1E+05 | | | 0.0005 | 0.05 | 5000 | 3 | 66 | 0.15 | 400 | | 1 | 3 |
| Mutant 2 | | | >250 | UL5 K356N | | | | | | | | | | |
| Mutant 6 | | | >1 | UL52 A897T | | | | | | | | | | |
| Example 5 | 3,E−08 | 2,E+09 | | | 3,E−08 | 0.005 | 50000 | 8 | 13 | 0.05 | 5000 | | 0.5 | 1.1 |
| Example 6 | 5,E−07 | 6,E+06 | | | 5,E−07 | 5,E−07 | 1,E+06 | 1.5 | 133 | 0.03 | 6660 | 7 nm un- | >0.5 | 0.5 |
| M1 + M2 | | | >125 | UL5 G352V | | | | | | | | compet- | | |
| | | | | | | | | | | | | itive | | |
| | | | | | | | | | | | | inhibition | | |
| Example 7 | 0.02 | 2500 | | | 0.02 | 0.02 | 3000 | 5 | >50 | 0.125 | 240 | 30 nm | 0.5 | 0.5 |
| M1 + M2 | | | >8 | UL5 G352V | | | | | | | | uncom- | | |
| | | | | | | | | | | | | petitive | | |
| | | | | | | | | | | | | inhibition | | |
| Acyclovir | 1 | 250 | 1 | | 125 | 4 | 125 | | | | | | 22 | 16 |
| | | | against | | | | | | | | | | | |
| | | | all | | | | | | | | | | | |
| | | | mutants | | | | | | | | | | | |
| Valtrex | | | | | | | | | | | | | 17 | 14 |

TABLE 2
Data for inhibition of the UL5-UL52-6xHis ATPase reaction
Titration of the enzymic activity with the compound mentioned as Example 6

| | Concentration of the compound designated Example 6 in | | | | | | | | |
|---|---|---|---|---|---|---|---|---|---|
| | the ATPase assay [μM] | | | | | | | | |
| | 100 | 20 | 4 | 0.8 | 0.16 | 0.032 | 0.0064 | 0.00128 | 0 |
| ATP [mM] | Concentration of liberated inorganic phosphate [mM] | | | | | | | | |
| 0.3 | 0.005 | 0.004 | 0.006 | 0.010 | 0.017 | 0.025 | 0.040 | 0.066 | 0.092 |
| 0.4 | 0.006 | 0.004 | 0.007 | 0.013 | 0.022 | 0.029 | 0.045 | 0.067 | 0.117 |
| 0.6 | 0.008 | 0.006 | 0.010 | 0.019 | 0.029 | 0.037 | 0.062 | 0.085 | 0.137 |
| 0.8 | 0.008 | 0.010 | 0.012 | 0.023 | 0.033 | 0.044 | 0.064 | 0.099 | 0.161 |
| 1 | 0.014 | 0.008 | 0.018 | 0.027 | 0.040 | 0.055 | 0.071 | 0.108 | 0.178 |
| 3 | 0.032 | 0.030 | 0.044 | 0.054 | 0.066 | 0.085 | 0.113 | 0.127 | 0.202 |

Preferred compounds are those which have in the viral replication assay described above an IC₅₀ (HSV-1 F/Vero cells) of less than 50 μM based on the fluorescence signal and, of course, more preferred compounds of an IC₅₀ of less than 25 μM, and the best compounds usually have an IC₅₀ of less than 10 μM.

The compounds according to the invention thus represent valuable active substances for the treatment and prophylaxis of diseases caused by herpes viruses, in particular herpes simplex viruses. Indications which can be mentioned by way of example are:

1) Treatment and prophylaxis of herpes infections, in particular herpes simplex infections in patients with pathological states such as herpes labialis, herpes genitalis, and HSV-related keratitis, encephalitis, pneumonia, hepatitis

2) Treatment and prophylaxis of herpes infections, in particular herpes simplex infections, in immunosuppressed patients (for example AIDS patients, cancer patients, patients with genetic immunodeficiency, transplant patients)

3) Treatment and prophylaxis of herpes infections, in particular herpes simplex infections, in neonates and infants

4) Treatment and prophylaxis of herpes infections, in particular herpes simplex infections, in herpes-positive, in particular herpes simplex-positive, patients for suppressing recurrence (suppression therapy)

d) In vivo activity/animal models

Animals

6-week old female mice, strain BALB/cABom, were purchased from a commercial breeder (Bomholtgard Breeding and Research Centre Ltd).

Infection

The animals were anaesthetized with diethyl ether (Merck) in a sealed glass vessel. 50 μl of a dilution of the virus stock (dose for infection 5×10⁴ Pfu) were introduced with an Eppendorf pippette into the nose of the anaesthetized animals. This dose leads to death of 90-100% of the animals due to a generalized infection with prominent respiratory and central nervous symptoms after on average between 5 and 8 days.

Treatment and Evaluation

6 hours after the infection, the animals were treated with doses of 0.1-100 mg/kg of body weight 3 times day at 7.00 h, 14.00 h and 19 h over a period of 5 days. The substances were dissolved in DMSO and resuspended in Tylose/PBS (Hoechst) (final concentration 1.5% DMSO, 0.5% Tylose in PBS).

After the last administration, the animals were observed further and the times of death were recorded.

Comparison of the survival plots revealed an ED₅₀ of about 0.5 mg/kg for HSV-2 for the compound of Example 7, for example, where ED₅₀ means that 50% of the animals survived with this dose.

The active substances of Examples 1 to 7 mentioned were synthesized as shown in the general scheme detailed hereinafter (FIG. 2shows the scheme for synthesizing the thiazolyl amides and FIG. 3shows the synthetic route to the thiazolylurea derivatives).

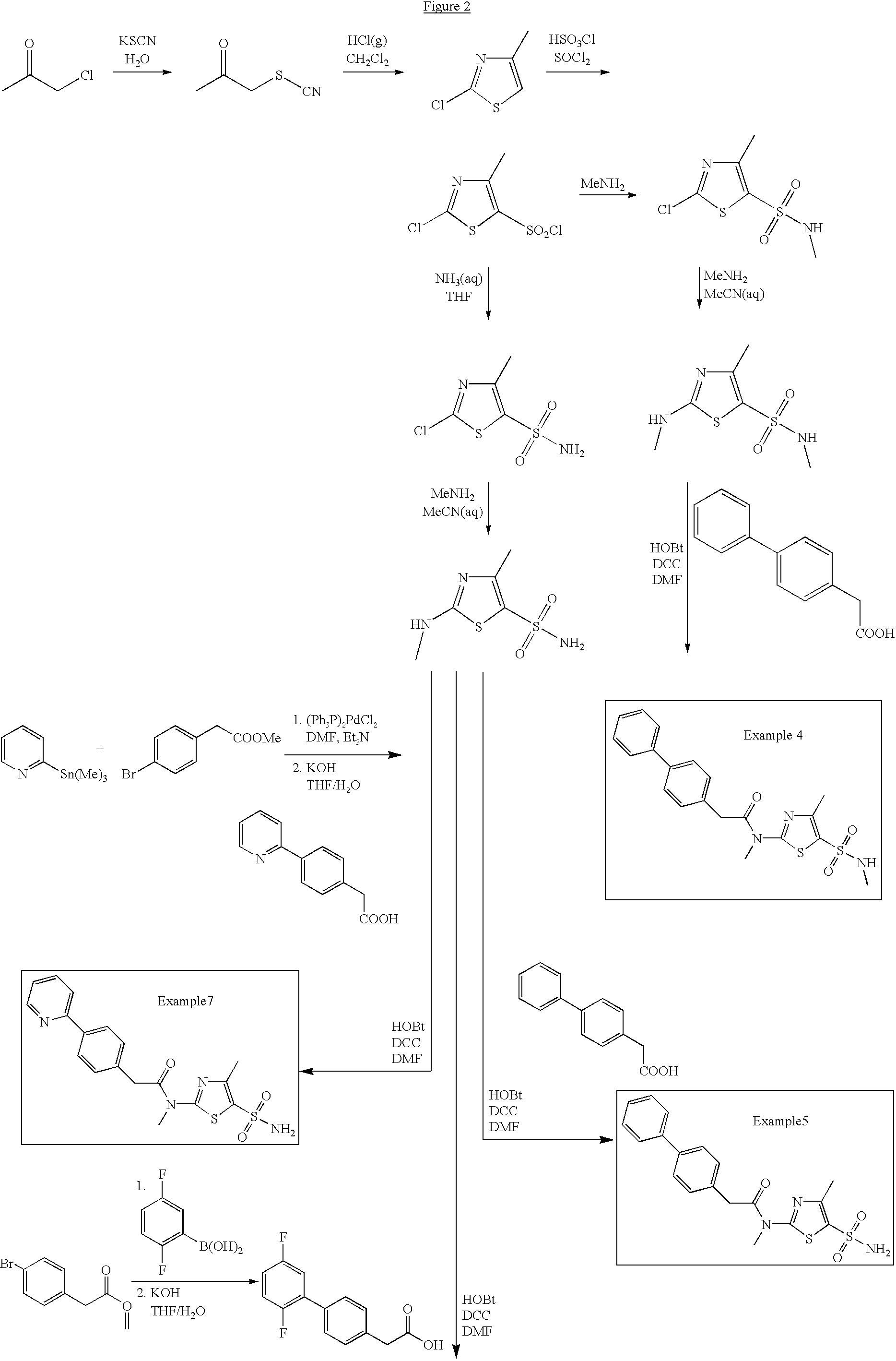

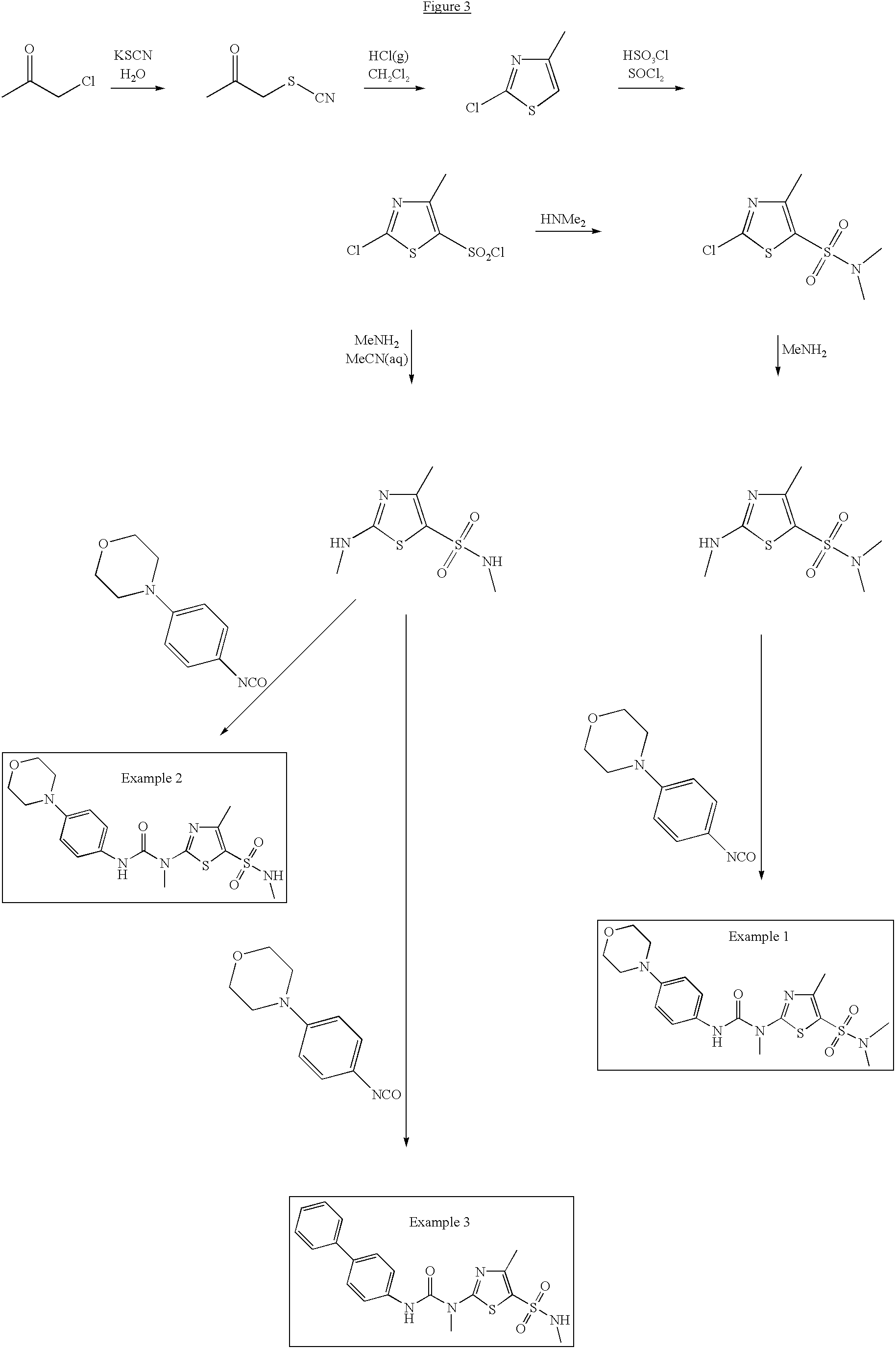

The active substance may have systemic and/or local effects. For this purpose, it can be administered in a suitable way, such as, for example, by the oral, parenteral, pulmonary, nasal, sublingual, lingual, buccal, rectal, transdermal, conjunctival or oticroute or as implant.

The active substance can be administered in suitable forms for these administration routes.

Suitable for oral administration are known administration forms which deliver the active substance rapidly and/or in a modified manner, such as, for example, tablets (uncoated and coated tablets, for example enteric coatings), capsules, sugar-coated tablets, granules, pellets, powders, emulsions, suspensions and solutions.

Parenteral administration can take place with avoidance of an absorption step (intravenous, intraarterial, intracardiac, intraspinal or intralumbar) or with inclusion of an absorption (intramuscular, subcutaneous, intracutaneous, percutaneous, or intraperitoneal). Administration forms suitable for parenteral administration are, inter alia, preparations for injection and infusion in the form of solutions, suspensions, emulsions, lyophilisates and sterile powders. Suitable examples for other administration routes are medicinal forms for inhalation (inter alia powder inhalers, nebulizers), nasal drops/solutions, sprays; tablets or capsules for lingual, sublingual or buccal administration, suppositories, preparations for the ears and eyes, vaginal capsules, aqueous suspensions (lotions, shaking mixtures), lipophilic suspensions, ointments, creams, milk, pastes, dusting powders or implants.

The active substances can be converted in a manner known per se into the administration forms mentioned. This takes place with use of inert, nontoxic, pharmaceutically suitable excipients. These include, inter alia, carriers (for example microcrystalline cellulose), solvents (for example liquid polyethylene glycols), emulsifiers (for example sodium dodecyl sulphate), dispersants (for example polyvinylpyrrolidone), synthetic and natural biopolymers (for example albumin), stabilizers (for example antioxidants such as ascorbic acid), colourings (for example inorganic pigments such as iron oxides) or masking flavours and/or odours.

It has generally proved to be advantageous on parenteral administration to administer amounts of up to about 0.01 to 100 mg/kg, preferably about 0.1 to 10 mg/kg, of body weight to achieve effective results. The amount on oral administration is about 0.1 to 50 mg/kg, preferably about 0.5 to 15 mg/kg, of body weight.

It may nevertheless be necessary where appropriate to deviate from the amounts mentioned, in particular as a finction of the body weight, administration route, individual behaviour towards the active substance, type of preparation and time or interval within which administration takes place.

1. Medicament containing uncompetitive inhibitors of the herpes helicase-primase.

2. Medicament according to claim 1, characterized in that the helicase-primase is at least 50% homologous with the helicase-primase of herpes simplex virus 1, based on the nucleotide sequence or the amino acid sequence, whichever is higher.

3. Medicament according to claim 1 or 2, characterized in that the virus is a herpes simplex virus.

4. Medicament according to claim 1, 2 or 3, characterized in that the compounds which bind simultaneously to the helicase and the primase subunits of the helicase-primase complex are selected.

5. Medicament according to any of claims 1 to 4, characterized in that the compounds inhibit the DNA-dependent NTPase activity of the helicase-primase.

6. Medicament according to any of claims 1 to 5, characterized in that the compounds have the ability to inhibit the replication of a herpes virus.

7. Medicament according to any of claims 1 to 6, characterized in that the compounds have the ability to inhibit the replication of a herpes virus in cell culture by at least 50% at a concentration of 10 μM.

8. Medicament according to any of claims 1 to 7, characterized in that those compounds have the ability to inhibit the replication of a herpes virus in cell culture by at least 50% at a concentration of ≦500 nM.

9. Medicament according to any of claims 1 to 8, characterized in that the herpes virus is PRV or BHV.

10. Method for identifying compounds with anti-herpes activity, characterized in that the compounds are tested for their ability to inhibit the enzymatic activity of helicase-primase, and those compounds which on competitively inhibit the helicase-primase enzyme complex are selected.

11. Compounds which are identified by methods according to claim 10.

12. Compounds according to claim 11, characterized in that the compound is not a nucleoside or nucleotide.

13. The use of a compound according to claim 11 or 12 for producing medicaments for the treatment and prophylaxis of herpes infections.

14. Medicaments or pharmaceutical formulations which contain compounds according to claims 11 or 12.

15. A method for the treatment and prophylaxis of herpes infections in mammals, characterized in that a therapeutically effective amount of the medicament or a pharmaceutical formulation according to claim 14 is administered to the mammal requiring such a therapy.

16. Medicaments according to any of claim 1 to 9, characterized in that the compounds have the following general structural formula (I)
Z—Y—A—X—SO₂NR¹R² (I)
in which
Z represents (C₆-C₁₀)-aryl, which optionally can be substituted by 1-3 substituents, selected from (C₁-C₆)-alkanoyl, (C₁-C₆)-alkoxy, (C₁-C₆)-alkyl, halogen, (C₁-C₆)-alkoxycarbonyl, nitrohalogen (C₁ -C₆)-alkyl, halogen(C₁-C₆)-alkoxy), amino(C₁-C₆)-alkylthio, hydroxy, carboxyl, carbamoyl, mono- or di-(C₁-C₆)-alkylaminocarbonyl, mono- or di-(C₁-C₆)alkanoylamino, (C₁-C₆)-alkoxycarbonylamino, (C₁-C₆)-alkylsulfoxy, C₁-C₆-alkylsulfonyl, tri-(C₁-C₆)-alkylsilyloxy, a 3- to 8-membered saturated or unsaturated, non-aromatic mono- or bicyclic heterocycle with up to 3 heteroatoms selected from the group S, N and/or O, which is optionally bonded via a nitrogen atom, and/or cyano,
or
Z represents a 5- to 6-membered aromatic heterocycle with up to 3 heteroatoms selected from the group S, N and/or O, which is optionally bonded via nitrogen atom, which is optionally substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halogen-(C₁-C₆)-alkyl, halogen-(C₁-C₆)-alkoxy, amino, (C₁-C₆)-alkylthio, hydroxy, carboxyl, carbamoyl, mono- or di-(C₁-C₆)-alkylamino-carbonyl, mono- or di-(C₁-C₆)-alkanoylamino, (C₁-C₆)-alkoxycarb-onylamino, (C₁-C₆)-alkylsulfoxy, (C₁-C₆)-alkylsulfonyl, a 3- to 8-membered saturated or unsaturated, non-aromatic mono- or bicyclic heterocycle with up to 3 heteroatoms selected from the group S, N and/or O, which is optionally bonded via a nitrogen atom, and/or cyano,
or
Z represents a 3- to 8-membered saturated or unsaturated, non-aromatic, mono- or bicyclic heterocycle, up to 3 heteroatoms selected from the group S, N and/or O, which is optionally bonded via a nitrogen atom, which can optionally be substituted by 1 to 3 substituents selected from oxo, halogen, hydroxy, (C₁-C₆)-alkoxycarbonyl, (C₁-C₆)-alkoxycarbonylamino, (C₁-C₆)-alkyl, halogen-(C₁-C₆)-alkyl and hydroxy-(C₁-C₆)-alkyl,
or
Z represents —OR¹⁹, —NR²⁰R²¹ or —CO—NR²²R²³,
wherein
R¹⁹ represents phenyl, which optionally can be substituted by a group of the formula —NR²⁴R²⁵, wherein
R²⁴ and R²⁵ are identical or different and represent hydrogen, (C₁-C₆)-alkyl or (C₁-C₆)-acyl,
or
R¹⁹ represents (C₁-C₆)-alkyl or (C₃-C₈)-cycloalkyl, which optionally can be substituted 1- to 3-fold by hydroxy and/or halogen,
R²⁰ and R²¹ are identical or different and represent hydrogen, carbamoyl, mono- or di-(C₁-C₆)-alkylaminocarbonyl, phenyl, (C₁-C₆)-acyl or (C₁-C₆)-alkyl,
wherein (C₁-C₆)-alkyl optionally can be substituted by (C₁-C₆)-alkoxy, (C₁-C₆)-acyl, phenyl or a 5- to 6-membered aromatic heterocycle with up to 3 heteroatoms selected from the group S, N and/or O,
wherein phenyl and aromatic heterocycle optionally can be substituted 1- to 3-fold identically or different by halogen and/or hydroxy,
R²² and R²³ are identical or different and represent hydrogen or (C₁-C₆)-alkyl,
Y represents (C₆-C₁₀)-aryl, a 5- to 6-membered aromatic heterocycle, a 5- to 6-membered aromatic benzo-fused heterocycle, a 3- to 8-membered saturated or unsaturated, non-aromatic heterocycle which is optionally bonded via a nitrogen atom, and which optionally can be substituted with 1 to 3 substituents selected from the group consisting of
halogen, (C₁-C₆)-alkoxy, (C₁-C₆)-alkoxycarbonyl, (C₁-C₆)-alkylthio, hydroxy, carboxyl, partially fluorinated (C₁-C₆)-alkoxy with up to 6 fluorine atoms, (C₁-C₆)-alkyl,
A represents a group of the formula —CR^{4′}R^{5′}—C(O)—CR⁴R⁵—, —CR^{4′}R^{5′}—C(O)—NR⁹—, —NR^{9′}—C(O)—CR⁴R⁵—or —NR^{9′}—C(O)—NR⁹—,
in which
R⁴, R^{4′}, R⁵, R^{5′}, R⁹ and R^{9′} are identical or different and represent hydrogen, (C₁-C₆)-alkyl, (C₂-C₆)-alkenyl or (C₃-C₈)-cycloalkyl,
X represents (C₆-C₁₀)-aryl, a 5- to 6-membered aromatic heterocycle, a 5- to 6-membered aromatic benzo-fused heterocycle, a 3- to 8-membered saturated or unsaturated, nonaromatic heterocycle which is optionally bonded via a nitrogen atom,
R¹ and R² are identical or different and represent hydrogen, (C₁-C₆)-alkyl or (C₃-C₈)-cycloalkyl,
where the A—X—SO₂ angle is 153°±15%, and the compound has a molecular mass of less than 500 g/mol,
and the salts thereof.
